(19) United States
(12) Patent Application Publication
(10) Pub. No.: US 2026/0026711 A1
(43) Pub. Date: Jan. 29, 2026

(54) Detect and Recognize Human Kinetic Movement electronically

(71) Applicants: Padmanabaiah Srirama, Glen Allen, VA (US); Satya Sai Chigurupati, O'Follon, MO (US); Padmavathi Jasti, O'Follon, MO (US); Akhil Srirama, Glen Allen, VA (US); Sravan Morishetty, Glen Allen, VA (US); Narayana Reddy Mara, Glen Allen, VA (US); Venkata Ramana Moorthy Bonala, Hyderabad (IN)

(72) Inventors: Padmanabaiah Srirama, Glen Allen, VA (US); Satya Sai Chigurupati, O'Follon, MO (US); Padmavathi Jasti, O'Follon, MO (US); Akhil Srirama, Glen Allen, VA (US); Sravan Morishetty, Glen Allen, VA (US); Narayana Reddy Mara, Glen Allen, VA (US); Venkata Ramana Moorthy Bonala, Hyderabad (IN)

(73) Assignee: Fit Well Technologies, Glen Allen, VA (US)

(21) Appl. No.: 18/784,779

(22) Filed: Jul. 25, 2024

(51) Int. Cl. A61B 5/11; A61B 5/00; A61B 5/024; G16H 10/60; G16H 40/67

(52) U.S. Cl. CPC A61B 5/1118; A61B 5/02405; A61B 5/6802; G16H 10/60; G16H 40/67

(57) ABSTRACT

The present invention pertains to an advanced method for automatically detecting and recognizing wellness activities and kinetic movements using accelerometer data from wearable devices. This method involves creating a user profile, linking the user to their wearable device via specific identifiers (make, type, and unique device ID), and processing accelerometer data to develop and continuously update a supervised machine learning model. This model accurately identifies and classifies various physical activities by utilizing detailed device information and user preferences. Designed for scalability, the system integrates new wearable devices and models as they emerge. It is particularly effective in tracking activities across diverse environments, including homes and fitness centers. Moreover, it identifies and associates ICD-10 activity codes with the performed activities and integrates with Electronic Medical Records (EMR) systems. This method offers valuable applications for fitness centers, healthcare providers, government agencies, and payers interested in monitoring or promoting physical wellness through reliable activity tracking.

### DETAIL DESCRIPTION OF THE INVENTION

Various aspects of the illustrative embodiments will be described using terms commonly employed by those skilled in the art to convey the substance of their work to others skilled in the art. It should be understood that the present invention may be practiced with only some of the described aspects. Specific numbers, materials, and configurations are set forth to provide a thorough understanding of the illustrative embodiments. However, it will be apparent to one skilled in the art that the present invention may be practiced without these specific details. In some instances, well-known features are omitted or simplified to avoid obscuring the illustrative embodiments.

The invention provides a comprehensive system and method for managing fitness and wellness data using biometric systems, wearable devices, and machine learning models to detect, classify, and analyze physical activities in real-time or batch modes. This system integrates wearable accelerometer data with gym equipment usage and biometric authentication to provide detailed user activity profiles.

Building upon the system disclosed in U.S. Pat. No. 9,852,264 B1, which introduced a biometric reading device for fitness activity tracking, this invention expands the use of wearable devices (accelerometers) and integrates them with gym equipment to monitor and classify user activities. In this system, biometric authentication is performed using a device as described in U.S. Pat. No. 9,852,264 B1, allowing users to authenticate and register their presence before interacting with gym equipment or initiating fitness sessions.

### System Components:

1. User and Device Association:

Users register their wearable devices via the web portal, specifying the device's brand, type, unique identifier, and wearing location (e.g., left or right wrist). This association is critical for accurately collecting accelerometer data during fitness activities and synchronizing it with gym equipment. The wearable device is linked to the biometric device (as described in U.S. Pat. No. 9,852,264 B1) to authenticate the user and ensure that only registered users can participate in activities.

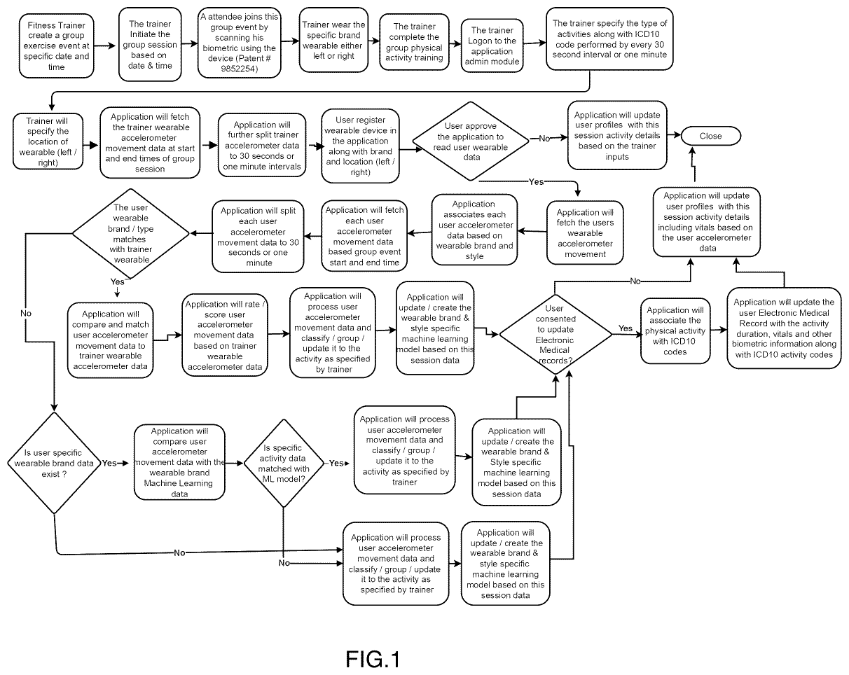

Provisional Patent Reference: This step is described in FIG. 1 of the provisional patent, where users authenticate via a biometric device before starting a gym session, and the accelerometer data from their wearable is logged for tracking.

2. Biometric Authentication with Gym Equipment:

The system integrates with gym equipment that is configured with a biometric device, as outlined in U.S. Pat. No. 9,852,264 B1. The user is authenticated using a biometric device before using the gym equipment. Gym equipment details (such as manufacturer, model, and associated fitness activity) are stored in the application, which is linked to the biometric device. This data is crucial for accurate fitness tracking and updating the user profile with the specific activities performed.

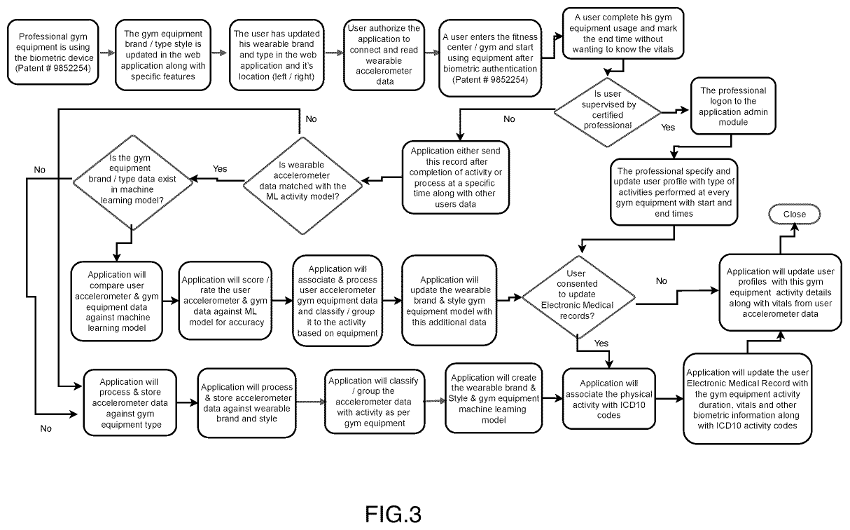

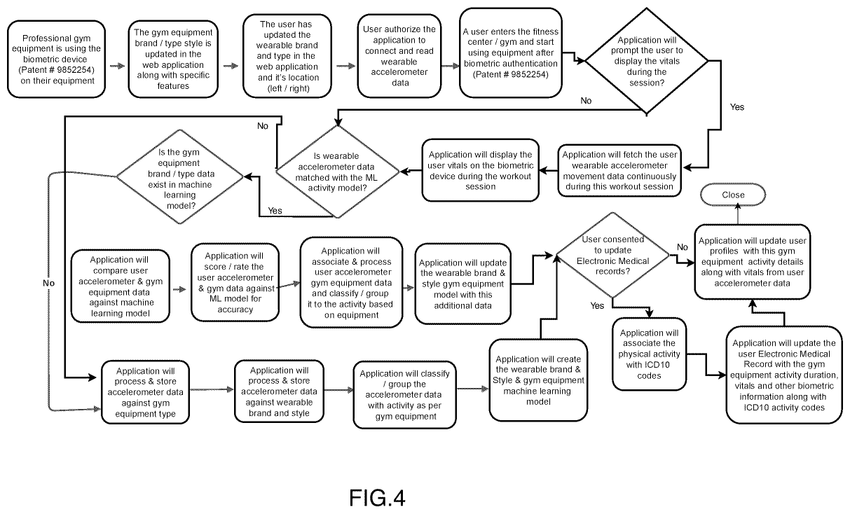

Provisional Patent Reference: This process is detailed in FIG. 3 of the provisional patent, where gym equipment is configured with biometric devices to log equipment usage, including start and end times, as well as any physical activities performed by the user.

### 3. Data Collection and Segmentation:

Data is continuously collected from both the wearable devices and gym equipment. For gym equipment, biometric authentication logs session start and end times, and accelerometer data is segmented into 30-second or 1-minute intervals for detailed analysis. This data segmentation helps the system accurately detect and classify various physical activities performed by the user.

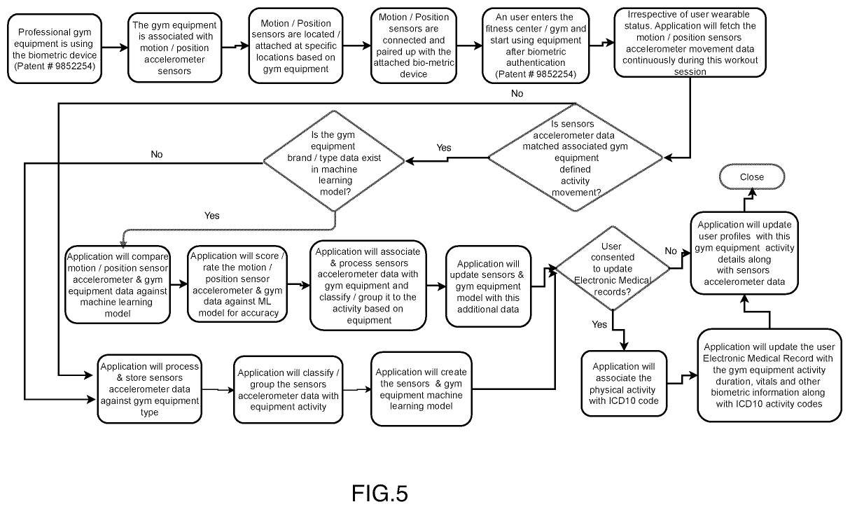

Provisional Patent Reference: FIGS. 4 and 5 in the provisional patent outline this process in detail, where user accelerometer data is collected and processed in real time or batch mode, depending on the configuration.

### 4. Data Processing and Machine Learning Models:

Once the accelerometer data is segmented, it is processed using a supervised machine learning model. The system compares the user's accelerometer data with pre-existing baseline models created by certified fitness professionals or trainers. If the user's accelerometer data matches an existing model, the system classifies the physical activity accordingly. If the data does not match, the system creates a new machine learning model for future reference.

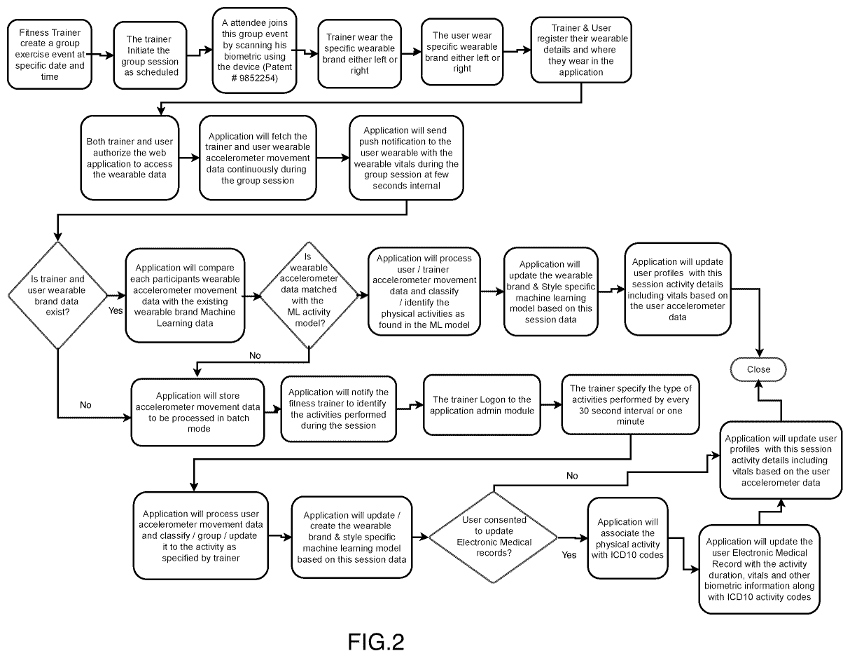

Provisional Patent Reference: This process is described in FIGS. 2, 3, and 5 of the provisional patent, where machine learning models are updated and created based on new activities or new wearable devices.

### 5. Activity Classification and Profile Updates:

The system classifies the physical activities performed by the user based on the data collected from the wearable device and gym equipment. The classified activities are then used to update the user's profile, including the type of activity, duration, intensity, and calories burned. This data is further refined based on the user's performance relative to baseline data.

Provisional Patent Reference: In FIG. 5 of the provisional patent, the system processes the accelerometer data, associates it with the gym equipment used, and updates the user's profile with detailed activity logs, including classifications and calories burned.

### 6. Handling New Devices and Activities:

When new wearables or new physical activities are introduced, the system dynamically adapts by creating new machine learning models. For example, if a user wears a new type of device or performs an activity not recognized by the system, the application creates a new machine learning model using data from the biometric system and wearable device.

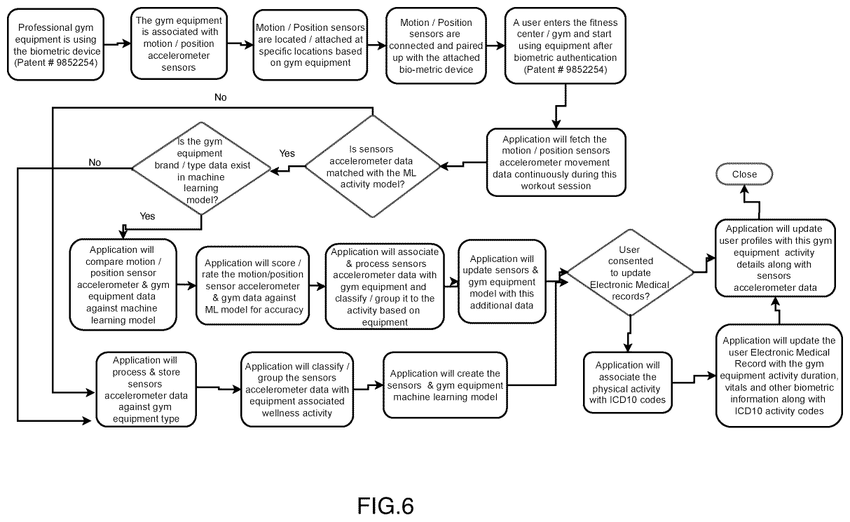

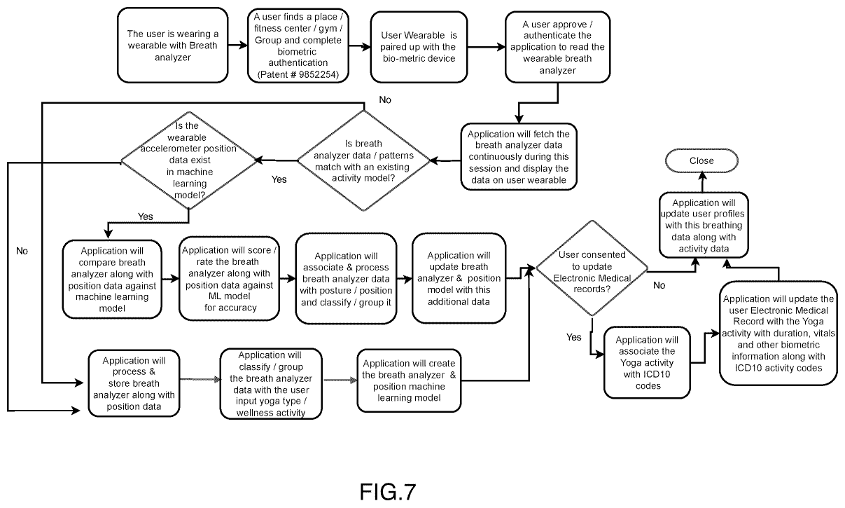

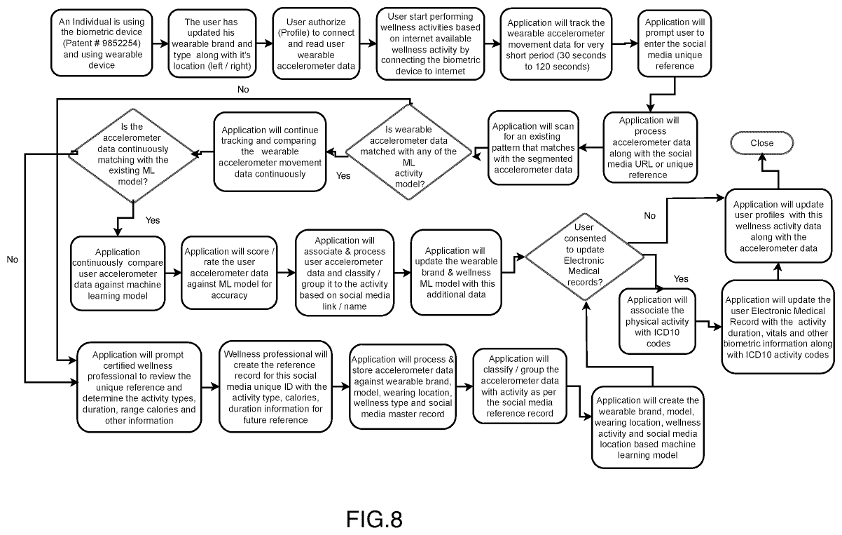

Provisional Patent Reference: FIGS. 4 and 6 of the provisional patent explain how the system adapts to new devices or activities by creating new machine learning models, which are stored for future use.

7. Real-Time vs. Batch Processing:

The system can operate in two modes: real-time processing and batch processing. In real-time mode, the user can view their vitals (heart rate, calories burned, etc.) during the session. The accelerometer and biometric data are processed and displayed on the user's device continuously. In batch mode, the data is processed at a specific time, such as after the session has ended.

Provisional Patent Reference: FIGS. 2 and 6 of the provisional patent describe these two modes, where real-time processing is used to display vitals during a session, and batch processing is employed to analyze data after the session ends.

8. Comparison with Trainer Data:

When users participate in fitness activities under the guidance of a certified trainer, their accelerometer data is compared with the trainer's baseline data. This comparison helps the system evaluate the user's performance and classify their activities more accurately. If the user's performance aligns with the trainer's data, the system updates the user's profile accordingly.

Provisional Patent Reference: In FIG. 3, the provisional patent describes how the system compares user accelerometer data with the trainer's data and scores the user's activity quality based on this comparison.

### 9. Machine Learning Data Model Creation:

If the system encounters new data points from the user's wearable device or gym equipment that do not match any existing models, it creates a new machine learning data model. This model includes the specific details of the wearable device (brand, location on the body) and gym equipment used during the session. These new models are stored for future activity recognition and classification.

Provisional Patent Reference: This process is explained in FIGS. 4, 5, and 6, where the system creates new machine learning models when encountering new devices or activities.

### CONCLUSION

The detailed invention description provided here ensures that the system captures every aspect of user authentication, data collection, and machine learning-driven activity classification. By integrating the biometric system from U.S. Pat. No. 9,852,264 B1 with real-time and batch accelerometer data processing, the invention offers a comprehensive solution for fitness activity tracking and profile management. The system's ability to handle new devices and activities through machine learning makes it adaptable and scalable for future fitness trends.

1: A method for detecting and recognizing fitness activities using wearable devices, comprising:
1. Establishing a user profile and associating the user profile with a wearable device through unique identifiers, including brand, type, and wearing location;
2. Collecting accelerometer data from both a fitness trainer and users during a fitness session;
3. Segmenting the collected accelerometer data into predefined time intervals of 30 seconds or 1 minute for detailed analysis;
4. Comparing the user's accelerometer data with the trainer's accelerometer data or an existing machine learning model for activity recognition;
5. Classifying the user's physical activities based on said comparison or machine learning model predictions;
6. Updating the user's profile with detailed session activity data and performance metrics, including activity type, duration, intensity, and calories burned.

2: The method of claim 1, further comprising adjusting machine learning models based on the wearable device's brand and model to improve activity recognition accuracy.

3: The method of claim 1, wherein the wearable device includes heart rate sensors, and the system uses heart rate data in conjunction with accelerometer data to refine activity classification.

4: The method of claim 1, further comprising analyzing multiple users' accelerometer data during a group fitness session to assess relative performance among users.

5: The method of claim 1, further comprising personalizing activity recognition based on the user's physical attributes, including height, weight, and age.

6: A method for generating a fitness report for an individual user using a wearable device, comprising:
1. Establishing a user profile and associating the user profile with a wearable device through unique identifiers, including brand, type, and wearing location;
2. Receiving consent from the user through a web portal to access and retrieve the user's wearable data;
3. Collecting accelerometer data from both fitness trainers and users during a fitness session;
4. Segmenting the accelerometer data into predefined time intervals of 30 seconds or 1 minute for detailed analysis;
5. Using a supervised machine learning model logged into an administrative module by the fitness trainer to specify the types of activities performed during the session;
6. Correlating accelerometer data with the wearable device worn on either the left or right wrist;
7. Determining whether the user's wearable device matches the trainer's wearable brand and type, and if not, verifying the existence of a machine learning model for the user's specific wearable device;
8. Rating and scoring the user's accelerometer data based on similarity to the trainer's data or machine learning model predictions;
9. Automatically updating and maintaining the user's fitness profile with session-specific and historical data, including calories burned, activity type, and duration;
10. Enhancing the accuracy and performance of the machine learning model through continuous learning and updating based on session data.

7: The method of claim 6, further comprising integrating additional sensor data from the wearable device, such as body temperature and skin conductivity, to enhance the fitness report.

8: The method of claim 6, wherein the fitness report is generated in multiple formats, including graphical, tabular, and textual summaries for user accessibility.

9: The method of claim 6, wherein the fitness report includes comparisons between previous fitness sessions to show progress over time.

10: The method of claim 6, further comprising alerting the user through a mobile application if their performance deviates significantly from expected fitness metrics.

11: A method for updating a user's electronic medical record (EMR) with the user's physical activities and associated ICD-10 codes, comprising:
1. Establishing a user profile and receiving consent from the user to update the electronic medical record;
2. Collecting wearable accelerometer data from the user during a fitness session;
3. Segmenting the collected accelerometer data into predefined time intervals of 30 seconds or 1 minute for analysis;
4. Comparing the user's accelerometer data with an existing machine learning model to recognize physical activities performed by the user;
5. Classifying the user's physical activities based on said comparison or model predictions;
6. Identifying the appropriate ICD-10 code for the physical activity performed by the user;
7. Converting the user's physical activity data into FHIR-compliant format for integration with the electronic medical record;
8. Updating the user's electronic medical record with physical activity data, including the ICD-10 code, date, time, and associated vital statistics collected from the wearable device;
9. Saving the user's electronic medical record and ensuring that historical data is updated with the latest session data;
10. Providing a historical record of physical activities in the user's electronic medical record for physician review.

12: The method of claim 11, further comprising generating new machine learning models customized for the user's specific wearable brand and model, based on session data collected from the wearable device during fitness activities, to improve the system's ability to recognize and classify future physical activities.

13: The method of claim 11, wherein the ICD-10 code is automatically selected based on the activity type and the user's medical history.

14: The method of claim 11, further comprising updating the user's EMR with heart rate data collected from the wearable device to track cardiovascular activity.

15: The method of claim 11, wherein the user's physical activity data is stored in both local and cloud-based storage for redundancy and security.

16: The method of claim 11, wherein the user's physician can remotely access the user's updated electronic medical record via a secure online portal for review.

17: The method of claim 11, for enhancing user engagement on a fitness web portal, comprising:
Providing additional content, including articles, advertisements, and links to discussion boards related to fitness and wellness, displayed alongside user fitness data and reports.
